(19) United States
(12) Patent Application Publication
(10) Pub. No.: US 2010/0112151 A1
(43) Pub. Date: May 6, 2010

(54) HIGH-VOLTAGE PULSED ELECTRICAL FIELD FOR ANTIMICROBIAL TREATMENT

(75) Inventors: Peter Bluestein, Vallhalla, NY (US); Mikhail Verbitsky, Stoughton, MA (US); Natalia Shibanova, St. Petersburg (RU); Rusian Yudin, St. Petersburg (RU); Rostislav Khorenyan, St. Petersburg (RU)

Correspondence Address: BANNER & WITCOFF, LTD.;and ATTORNEYS FOR CLIENT NO. 006943, 10 SOUTH WACKER DR., SUITE 3000, CHICAGO, IL, 60606, US

(73) Assignee: PepsiCo., Inc., Purchase, NY (US)

(21) Appl. No.: 12/609,802

(22) Filed: Oct. 30, 2009

Related U.S. Application Data
(60) Provisional application No. 61/111,577, filed on Nov. 5, 2008.

(51) Int. Cl. A23L 3/32; A61L 2/03

(52) U.S. Cl. 426/244; 422/22; 422/186.04

(57) ABSTRACT

Aspects of the invention relate to a device and method for non-contact inactivation of undesirable and/or harmful microorganisms in products using high-voltage nanosecond pulsed electrical field. In certain embodiments, a product may be packaged into a container which is made from a dielectric material and placed between electrodes to be processed by a pulsed electrical field. In certain embodiments, the electrodes, together with the container, may be placed into a treatment assembly filled with a high dielectric permeability media that allows the formation of a quasi-uniform electrical field inside the product and prevents the electrical breakdown of the dielectric material of the container. The electrodes may be connected to a high voltage generator, which forms nanosecond pulses that allow an electrical field of high intensity to penetrate the dielectric material of container walls and gaps between the electrodes and the container's walls to the product without significant energy losses.

The present application claims the benefit of U.S. provisional patent application No. 61/111,577, filed Nov. 5, 2008 and entitled “High-Voltage Pulsed Electrical Field for Antimicrobial Treatment,” the entire disclosure of which is hereby incorporated by reference.

### FIELD OF THE INVENTION

This invention relates to a method and system for antimicrobial treatment. In particular, this invention relates to a method and system for fluid media treatment to inactivate harmful microorganisms using high-voltage nanosecond pulsed electrical field.

### BACKGROUND

A high intensity pulsed electric field (“PEF”) may be employed for treating fluid medium, such as liquid products (including, but not limited to, liquid foods and medicines), to inactivate biocontamination, such as bacteria, fungi, spores etc. PEF inactivates microorganisms causing damage to their cell membranes or injuring their subcellular structure.

Conventional PEF processing systems include a pulsed high voltage generator and electrodes for creating an electric field in a treatment chamber. PEF processes use high voltage pulses to generate short duration pulsating electric fields in a product. The short duration of pulses is preferred to prevent undesirable heating of the treated product.

PEF systems generally require direct physical and electrical contact between the medium being treated and the electrodes during the treatment. Such systems typically generate a field strength within a range of 5-100 kV/cm and have a pulse duration in the range of about 0.1-100 microseconds.

However, using a 0.1-100 microseconds pulse duration may be less effective when attempting to treat packaged products (treatment of a medium not in direct contact with the electrodes) because of the high energy loss due to various reasons—e.g. the packaging materials and air gaps between electrodes and packaging may diminish the effect of the pulse. Additionally, high energy pulses may not be able to be applied to treat foods with high electrical conductivity because intensive electric current may cause electrical breakdown of the food and change its organoleptic properties.

### BRIEF SUMMARY

Aspects of the invention may overcome disadvantages in the prior art, provide devices and methods for non-contact antimicrobial treatment of packaged products, and prevent the electrical breakdown of dielectric packaging material, which may occur when a high voltage pulsed electrical field is applied. In certain aspects, this may be accomplished by creating a quasi-uniform electrical field of high intensity in products placed into dielectric containers of complex shape.

It will be appreciated by those skilled in the art, given the benefit of the following description of certain exemplary embodiments of the beverage and other beverage products disclosed here, that at least certain embodiments of the invention have improved or alternative formulations suitable to provide desirable taste profiles, nutritional characteristics, etc. These and other aspects, features and advantages of the invention or of certain embodiments of the invention will be further understood by those skilled in the art from the following description of exemplary embodiments.

### BRIEF DESCRIPTION OF THE DRAWINGS

The present disclosure is illustrated by way of example and not limited in the accompanying figures in which like reference numerals indicate similar elements and in which:

FIG. 1 shows an illustrative pulsed electric field treatment device according to some embodiments of the present invention;

FIG. 2A depicts an illustrative chart of the output of the high voltage generator in some embodiments of the invention;

FIG. 2B depicts an illustrative chart of the pulse packet formed on electrodes in some embodiments of the invention;

FIG. 3 shows an illustrative application of a pulsed electric field treatment device to a conveyer-escalator type filling line according to aspects of the invention; and

FIG. 4 shows an illustrative application of a pulsed electric field treatment device to a conveyer-rotator type filling line according to aspects of the invention.

FIG. 5 shows an illustrative flow chart of a method that may be used to treat a product in a container according to aspects of the invention.

FIG. 6A shows an illustrative complex electrode shape according to aspects of the invention.

FIG. 6B shows a second illustrative complex electrode shape according to aspects of the invention.

### DETAILED DESCRIPTION

In accordance with various aspects of the disclosure, a method and system for treatment of a product to inactivate harmful microorganisms using a high-voltage nanosecond pulsed electrical field is disclosed. The product to be treated can be any of various items including products containing oil and/or water, foodstuffs, beverages, pharmaceuticals, nutraceuticals, etc. The products may be packaged in many types of containers including bottles, which may be made from a polymer such as polyethylene terephthalate. In the following description of the various embodiments, reference is made to the accompanying drawings, which form a part hereof, and in which are shown by way of illustration various embodiments in which the invention may be practiced. Certain embodiments are described as “illustrative” or “exemplary,” which indicates that these embodiments are just examples of potential embodiments and are not to be interpreted as preferred or sole embodiments. It is to be understood that other embodiments may be utilized and structural and functional modifications may be made without departing from the scope of the present invention.

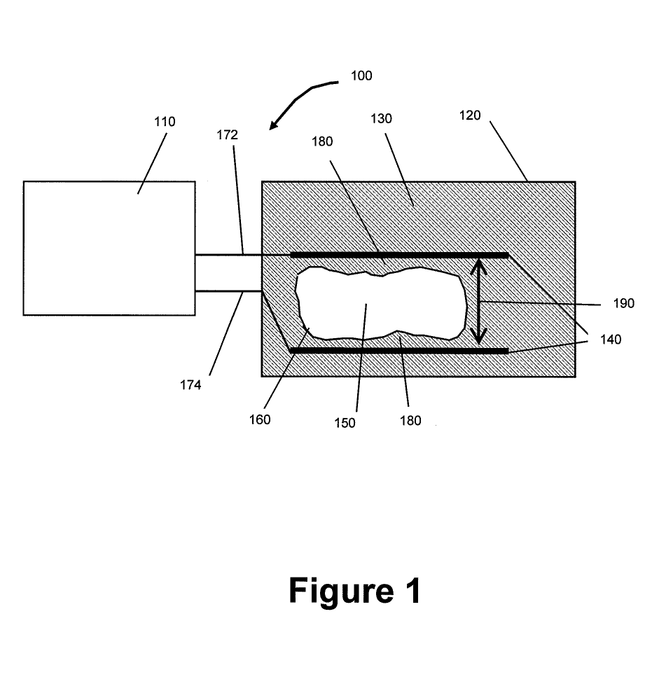

FIG. 1 depicts an exemplary pulsed electric field treatment system 100 for processing products. Treatment system 100 may include high voltage generator 110, treatment assembly 120, and one or more electrodes 140. Treatment assembly 120 may be filled with a medium 130 having high dielectric permeability, generally higher than approximately 30. In some embodiments, medium 130 may be de-ionized water, generally having a high dielectric permeability of approximately 80. The system may be used to treat a product 150, which may be contained by a product container 160.

One embodiment depicted in FIG. 1 may include two electrodes 140 that may be connected to generator 110 via wires 172, 174. In some embodiments, one of electrodes 140 may be grounded. In at least one embodiment, a space 190 may be formed between the electrodes 140 and may form a treatment zone where a product may be treated by an electrical field.

The container 160 containing product 150 may be made of a dielectric material. The container 160 may have regular or complex shape. In certain embodiments, the thickness of the walls of container 160 may be in the range of 50 micrometers to 1 millimeter. In some embodiments, the thickness of the walls of container 160 may be between 50 and 400 micrometers. In aspects of the invention, limiting the thickness of the walls of container 160 may minimize energy losses in the walls of container 160.

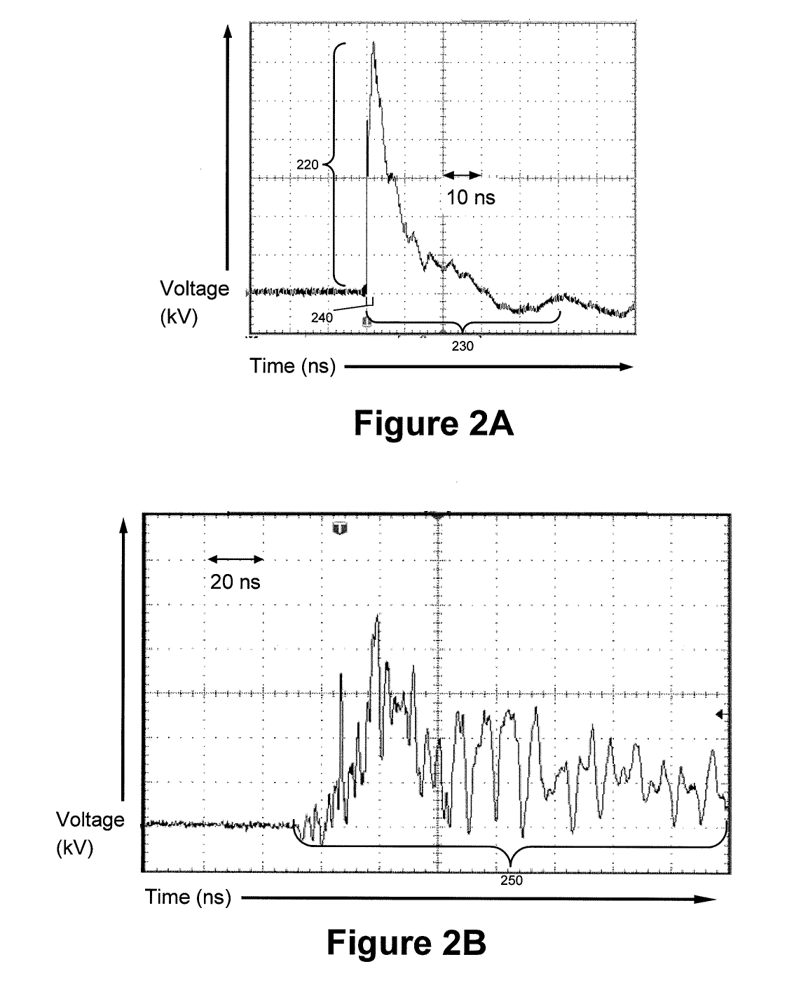

Generator 110 may produce high-voltage single-polarity or dual-polarity electrical pulses. Exemplary amplitudes 220 of such pulses may range from 100 to 1000 kilovolts as depicted in FIG. 2A. In certain embodiments, the output voltage generated by generator 110 may be selected by determining the electrical field strength desired inside product 150 to inactivate undesirable and/or harmful microorganisms. Energy losses that may occur due to container 160 thickness, gaps 180 between electrodes 140 and container 160, size of container 160, and product's 150 properties may be taken into account in determining the electrical field strength desired and/or the output voltage to be generated. In some embodiments, the electrical field strength inside product 150 is in the range of 10 to 100 kilovolts/centimeter.

In one embodiment, the pulse generated by generator 110 may have a duration 230 of approximately 5 to 50 nanoseconds and a rise time 240 of approximately 1 nanosecond. The nanosecond rise time may generate an electrical field of high intensity that may be delivered to the product through the dielectric material of the walls of container 160 and through the gaps between electrodes 140 and the walls of container 160 without significant losses. Pulses having short duration may avoid undesirable heating and may reduce the cost of running generator 110 due to reduced energy consumption during treatment of product 150.

The number of pulses, pulse frequency, shape, and the input pulse voltage may vary based on the type of product 150 being treated, the type of microorganism contamination for which product 150 is being treated, and the required time of treatment. In some embodiments, between 1 and 10,000 pulses may be generated with an input pulse voltage in the range of 100 to 1000 kilovolts. In certain embodiments, the frequency of pulses generated may be between 1 and 10,000 Hz.

Electrodes 140, together with the container 160 may be placed into treatment assembly 120, which may be filled by medium 130 having high dielectric permeability. Electrodes 140 and container 160 do not need to be in direct contact, allowing a gap 180.

Electrodes 140 may be made of various materials and may be of many shapes and sizes. In some embodiments, electrodes 140 are composed of a metal material. In one embodiment, electrodes 140 may be made of stainless steel. Stainless steel electrodes 140 may reduce electron emission from the metal to the surrounding media 130 when subjected to an electric field. Reduction of electron emission may minimize the probability of the electrical breakdown of the dielectric material of container 160.

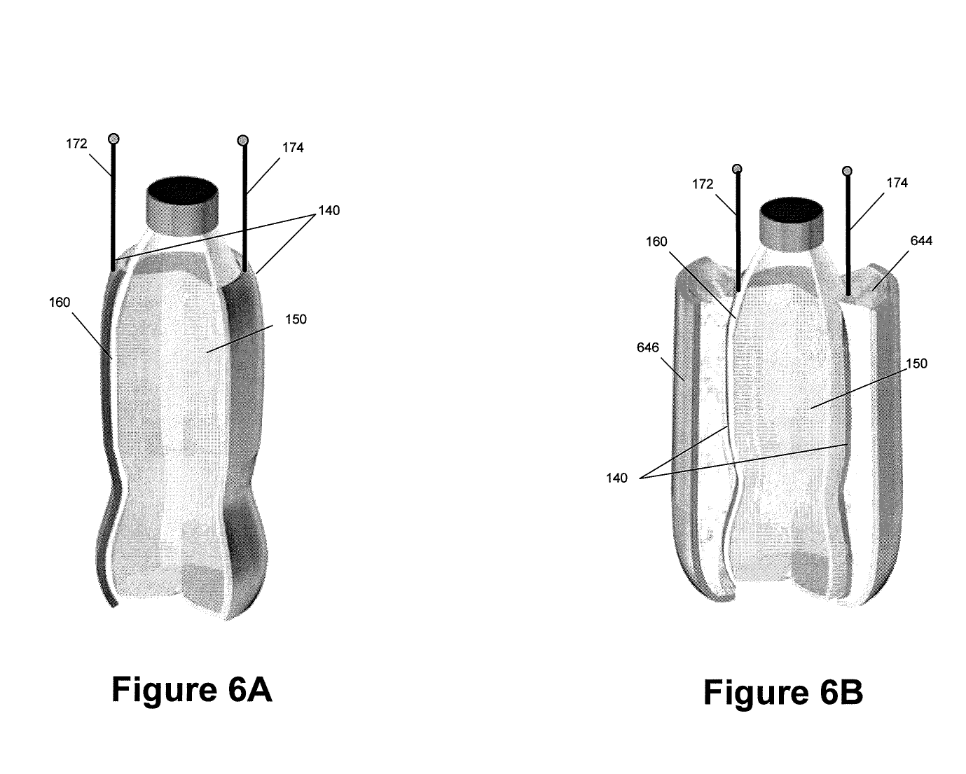

In certain embodiments, electrodes 140 may be flat plates. This shape may provide a quasi-uniform electrical field inside product 150. The size of electrodes 140 and inter-electrode space 190 may vary depending on the size of container 160. In other embodiments, electrodes may have a complex shape as depicted in FIGS. 6A and 6B. The embodiment depicted in FIG. 6A shows electrodes 140 having a complex shape similar to the exterior shape of container 160. In some embodiments, electrodes 140 may be of an exact shape to match the shape of container 160 such that electrodes 140 are in direct contact with container 160. In other embodiments, electrodes 140 may not be in direct contact with container 160 such that there is a gap between electrodes 140 and container 160.

Similarly, in some embodiments depicted in FIG. 6B, electrodes 140 may directly contact container 160 whereas other embodiments may leave a gap between electrodes 140 and container 160. The embodiment depicted in FIG. 6B employs a sponge 644 or sponge-like material. In such embodiments, electrodes 140 may be attached to a surface of sponge 644 and electrodes 140 may be composed of a flexible metalized film. Flexible electrodes 140 attached to a sponge 644 may allow the electrodes to form a complex shape similar to the shape of the exterior of container 160. In some embodiments, the assembly may also include an electrode holder 646 to which sponge 644 may be attached. Electrode holder 646 may provide a firm surface to grip or attach to the rest of the assembly. There are many other possible electrode configurations. The embodiments depicted in FIGS. 6A and 6B are merely illustrative of two possible embodiments. Other embodiments may include depositing electrodes on the surface of container 160 such as part of a bottle label or design, embedding electrodes into aspects of the treatment assembly (such as attaching electrodes to portions of the assembly that grip or transfer container 160, etc. In other embodiments, at least one of electrodes 140 may have a knife-point edge or be a point-source electrode.

In some embodiments, electrodes 140 may have a length comparable to the pulse 230 wavelength. In such embodiments, numerous pulses 230 may be reflected from both ends of electrodes 140 and form a pulse packet 250 within product 150 as shown in FIG. 2B. The formation of pulse packet 250 may result in increasing efficacy of the inactivation of harmful microorganisms by affecting the microorganisms' membranes or injuring their subcellular structure. As can be seen in FIG. 2B, the formation of pulse packet 250 within product 150 from a single generated pulse 230 (as depicted in FIG. 2A) may increase the number of voltage swings that product 150 is subjected to as compared to a traditional single pulse. Subjecting product 150 to an increased number of voltage swings may assist in breaking down the organisms' membranes and ripping the organisms apart. Therefore, when using electrodes 140 with a length approximately equal to the pulse wavelength, which may allow for favorable conditions to obtain resonance and maintain maximum pulse amplitude, each pulse 230 generated by generator 110 may result in an electrical field present in product 150 that includes a group of pulses, or a pulse packet 250, without requiring additional energy from generator 110. Variation in the length of electrodes 140 may provide different combinations of pulse interactions in the pulse packet 250 (i.e., different amplitudes, frequencies, and rise times).

In accordance with different sized product containers, certain embodiments may have a space between electrodes 140 (the “treatment zone” 190 or inter-electrode space) ranging from approximately 1 to approximately 10 centimeters. For containers 160 made of different dielectric materials, different gaps 180 between electrodes 140 and container 160 may be used. In some embodiments, gaps 180 may be between 0.1 millimeters and 2 centimeters, depending on the electrical breakdown properties of the dielectric material of container 160, the thickness of the walls of container 160, and the shape of container 160. In one embodiment, treatment of the packaged product 150 may simultaneously inactivate microorganisms' in product 150 and in the inner surface of container 160. In such embodiments, the need to separately disinfect container 160 may be eliminated and the total cost of production may therefore be reduced.

In some embodiments, treatment assembly 120 may be filled with a medium 130. In one embodiment, medium 130 may have a high dielectric permeability, which may assist in: (i) forming a quasi-uniform electrical field in all parts of the product 150, which is placed into container 160 (container 160 may be of a complex or regular shape); (ii) avoiding the electrical breakdown of the dielectric material of container 160 by diminishing the effect of electrical voltage concentrators, which generally exist on electrodes' 140 surface, (iii) passing an electrical field of high intensity to product 150 through the gaps between electrodes 140 and the walls of container 160 without significant losses. Embodiments including a medium 130 having a high dielectric permeability may result in less significant losses than embodiments including a medium having low dielectric permeability, such as air gaps. In certain embodiments, medium 130 may also have low conductivity.

### Exemplary Process

In an exemplary method of treating a product with a pulsed electrical field to inactivate biocontamination in the product or the interior of the product container, a treatment assembly may be filled with a medium 130. In some embodiments, a container meant to hold a product may be sterilized in step 510 and the product may be placed into the container in step 520. Alternatively, the product may be placed into the container in step 520 and the container may be sterilized 510 after the product is in the container. The container may then be sterilized separately from the product in step 510, or, alternatively, the container may be sterilized when an electrical pulse is generated in step 540, described below. In step 530, the container may be placed into the treatment assembly. The container may be placed in the treatment assembly in any of a variety of ways, including, for example, manually placing the container in the treatment assembly, placing the container on a conveyor line, etc. An electrical pulse may be generated in step 540. The electrical pulse may be generated using a high voltage generator or any other system capable of producing an electrical pulse with the desired characteristics, such as field strength, duration, etc. In certain embodiments, a series of electrical pulses may be generated. In some embodiments, the wavelength of the pulse generated may be comparable to the length of the electrodes such that a pulse packet is generated.

### EXAMPLES

The following examples are specific embodiments of the present invention but are not intended to limit it.

### Example 1

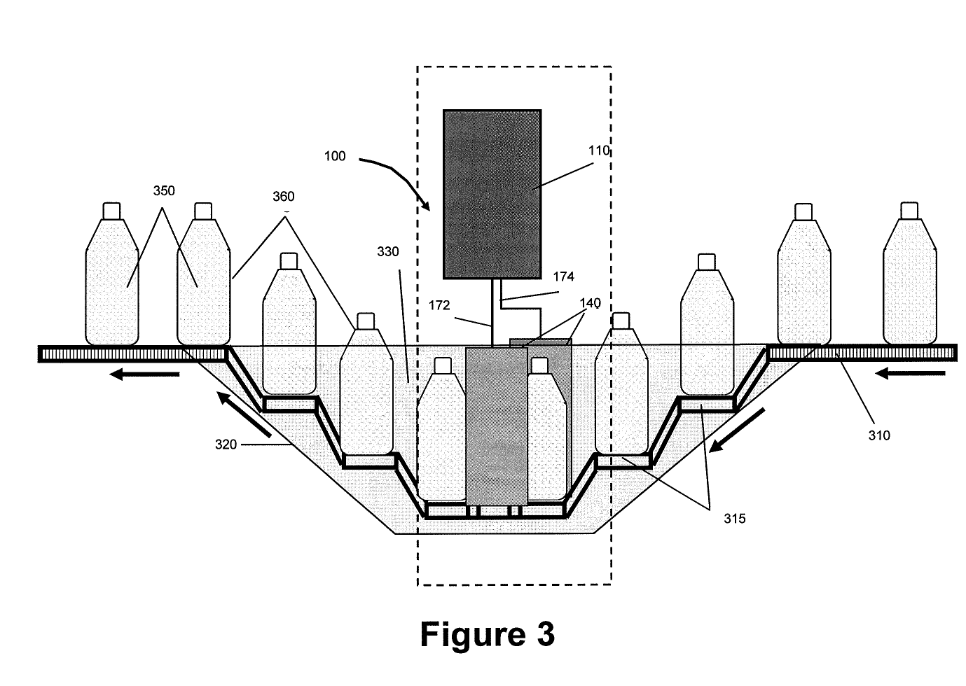

FIG. 3 depicts one possible embodiment of the present invention that may be integrated with a conveyer line 310. Conveyor line 310 may be used for filling container 360 with product 350. The example shown in FIG. 3 depicts beverages as product 350 and bottles as container 360. The pulsed electrical field treatment device 100 may be placed along conveyer line 310. Treatment assembly 320 may include an area that may be filled with a medium 330. In some embodiments, medium 330 may be a medium having a high dielectric permeability. In one embodiment, medium 330 may be de-ionized water. Conveyor 310 may transport product containers 360 to treatment assembly 320. In one embodiment, product containers 360 may be bottles. In certain embodiments, product containers 360 may be polyethylene terephthalate (PET) bottles. Optionally, a segment of conveyer line 310 may be modified to create a conveyer-escalator 315. Product containers 360 may be transported along conveyor line 310 and, when transported to conveyer-escalator 315, product containers 360 may enter treatment assembly 320. As product 350 in product containers 360 pass through treatment assembly 320 along conveyor line 310, product 350 and container 360 may pass between two electrodes 140. At certain time intervals, product 350 and container 360 may be treated by electrical field pulses generated between electrodes 140. High voltage pulses may be transmitted to electrodes 140 via wires 172, 174 from generator 110. As a result, undesirable and/or harmful microorganisms in product 350 and on the inner surface of container 360 may be inactivated.

### Example 2

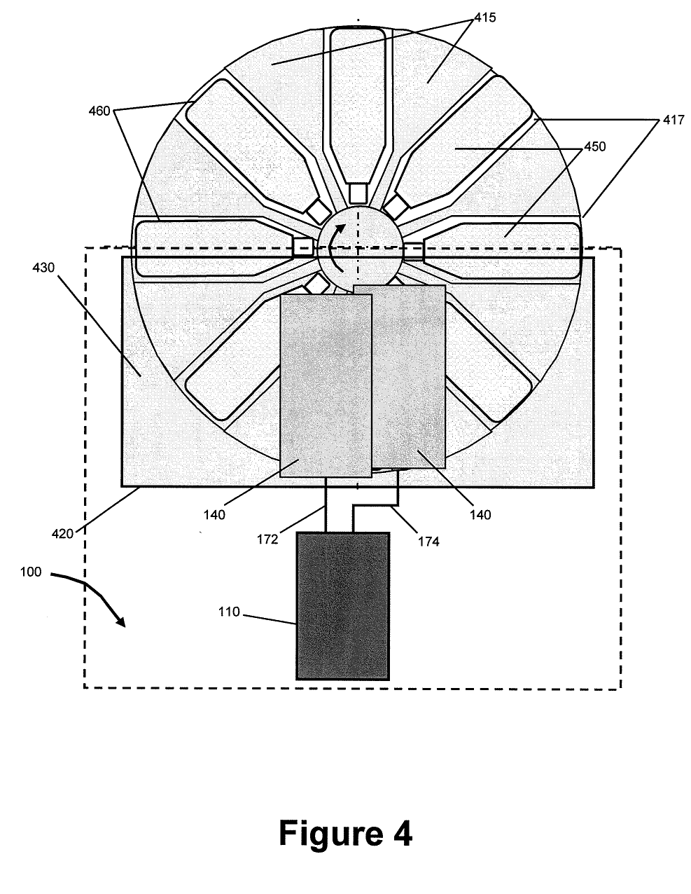

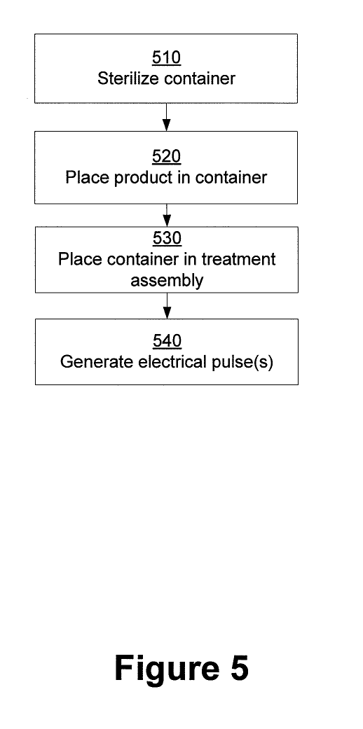

FIG. 4 depicts another possible embodiment of the present invention that may be integrated with a conveyer line (not shown). Conveyor line may be used for filling container 460 with product 450. In the embodiment depicted in FIG. 4, conveyer line may include conveyer-rotator 415. In one embodiment, product containers 460 may be bottles and product 450 may be beverages. In certain embodiments, product containers 460 may be PET bottles. The pulsed electrical field treatment device 100 may be placed along conveyor line. Treatment assembly 420 may include an area that may be filled with a medium 430. In some embodiments, medium 430 may be a medium having a high dielectric permeability. In one embodiment, medium 430 may be de-ionized water. Conveyor may transport product containers 460 to treatment assembly 420. Container 460 may then enter a cell 417 of conveyer-rotator 415, which may then transport container 460 to treatment assembly 420. At certain time intervals, product 450 and container 460 may be treated by electrical field pulses generated between electrodes 140. High voltage pulses may be transmitted to electrodes 140 via wires 172, 174 from generator 110. As a result, undesirable and/or harmful microorganisms in product 450 and on the inner surface of container 460 may be inactivated. Variations of the described embodiment are also possible. For example, in some embodiments, electrodes 140 may be connected to or a part of cell 417, such that portions of the interior lining of cell 417 may constitute electrodes 140 (one portion constituting a ground electrode and another portion constituting a charged electrode).

Aspects of the invention have been described in terms of illustrative embodiments thereof. Numerous other embodiments, modifications and variations within the scope and spirit of the appended claims will occur to persons of ordinary skill in the art from a review of this disclosure. For example, one of ordinary skill in the art will appreciate that the steps illustrated in the figures may be performed in other than the recited order, and that one or more steps illustrated may be optional in accordance with aspects of the disclosure. Given the benefit of the above disclosure and description of exemplary embodiments, it will be apparent to those skilled in the art that numerous alternative and different embodiments are possible in keeping with the general principles of the invention disclosed here. Those skilled in this art will recognize that all such various modifications and alternative embodiments are within the true scope and spirit of the invention.

What is claimed is:

1. A method for treating a product comprising:
filling a treatment assembly with a medium, wherein the treatment assembly includes a plurality of electrodes connected to a high voltage generator;
placing a container between the plurality of electrodes, wherein the container contains the product; and
generating an electrical pulse using the high voltage generator, wherein the electrical pulse has a duration of less than 1 microsecond.

2. The method of claim 1, wherein the length of at least one of the plurality of electrodes is approximately equal to the wavelength of the electrical pulse and wherein the electrical pulse forms a pulse packet inside the product.

3. The method of claim 2, wherein the container is made from a dielectric material.

4. The method of claim 2, wherein the container has a complex shape.

5. The method of claim 2, wherein the medium has a dielectric permeability greater than 30.

6. The method of claim 5, wherein the medium is de-ionized water.

7. The method of claim 2, wherein the electrical pulse has a duration of between 1 and 200 nanoseconds and wherein the electrical pulse has a rise time between 0.5 and 100 nanoseconds.

8. The method of claim 1, wherein the product contains one or more chemical preservatives.

9. The method of claim 2, wherein the product is a foodstuff.

10. The method of claim 2, wherein the product is a beverage.

11. The method of claim 2, wherein the container is made from a polymer.

12. The method of claim 2, wherein the walls of the container have a thickness in the range of 50 to 1000 micrometers.

13. The method of claim 2, wherein there is a gap between the plurality of electrodes and the container, the gap having a range of 0.1 to 20 millimeters.

14. The method of claim 2, wherein the plurality of electrodes are comprised of stainless steel.

15. The method of claim 2, wherein at least one of the plurality of electrodes is of complex shape.

16. The method of claim 2, wherein one of the plurality of electrodes is a point-source electrode.

17. The method of claim 2, wherein the plurality of electrodes are a part of the container.

18. The method of claim 2, further comprising heating the medium to a temperature between 20 and 99 degrees Celsius.

19. The method of claim 18, wherein the medium is heated to a temperature between 30 and 60 degrees Celsius.

20. The method of claim 18, wherein the medium is heated to a temperature between 48 and 52 degrees Celsius.

21. The method of claim 2, wherein the peak voltage of the electrical pulse has a range of between 100 and 1000 kilovolts.

22. The method of claim 2, wherein the plurality of electrodes are separated from each other by a range of 1 to 10 centimeters.

23. The method of claim 2, wherein the electrical pulse generates an electrical field inside the product having an electrical field strength ranging from 10 to 100 kilovolts per centimeter.

24. The method of claim 2, wherein the electrical pulse has a frequency range from 0.5 to 10,000 Hertz.

25. The method of claim 1, wherein the electrical pulse has a duration of less than 300 nanoseconds.

26. A container for holding a product comprising at least one electrode configured to be connected to a high voltage generator.

27. The container of claim 26, wherein the container is configured to contain a foodstuff.

28. The container of claim 26, wherein the electrode is comprised of a flexible foil.

29. The container of claim 28, wherein the electrode is integrated with a label for the container.

30. The container of claim 26, wherein the length of the at least one electrode is approximately equal to the wavelength of an electrical pulse to be applied to the at least one electrode.
